(19) United States
(12) Patent Application Publication
(10) Pub. No.: US 2013/0071487 A1
(43) Pub. Date: Mar. 21, 2013

(54) XENON-BASED INHALABLE DRUG FOR PREVENTING ADDICTION RELAPSES IN HUMANS

(71) Applicants: Baptiste Bessiere, Issy les Moulineaux (FR); Jan Pype, Herne (BE)

(72) Inventors: Baptiste Bessiere, Issy les Moulineaux (FR); Jan Pype, Herne (BE)

(73) Assignee: L'Air Liquide, Societe Anonyme pour l'Etude et l'Exploitation des Procedes Georges Claude, Paris (FR)

(21) Appl. No.: 13/698,813

(22) PCT Filed: May 9, 2011
(86) PCT No.: PCT/FR11/51038

§ 371 Date: Nov. 19, 2012

(30) Foreign Application Priority Data
Jun. 3, 2010 (FR) 1054333

(51) Int. Cl. A61K 33/00; A61P 25/36; A61P 25/32; A61P 25/30

(52) U.S. Cl. 424/600

(57) ABSTRACT

The invention relates to a gas composition containing xenon gas for use in preventing the relapse of a patient who has been weaned from at least one psychotropic product which resulted in said patient becoming habituated, the xenon being administered to the patient by inhalation. The composition of the invention contains an effective volume proportion of xenon, in particular from 5 to 70% by volume of xenon. DRAWING: FIG. 5: Nombres de pression sur le levier/session Number of presses on the lever/session Levier respectif inactif Respective inactive lever rechute relapse

### CROSS REFERENCE TO RELATED APPLICATIONS

This application is a 371 of International PCT Application PCT/FR2011/051038, filed May 9, 2011, which claims priority to French Patent Application No. 1054333 filed 3 Jun. 2010, the entire contents of which are incorporated herein by reference.

### BACKGROUND

The present invention relates to a gaseous composition, that is to say a gaseous medication, based on gaseous xenon administrable by inhalation in order to prevent, avoid or reduce susceptibility to relapse in a patient treated for dependence or addiction, particularly to one or more drugs and/or alcohol, that is to say including human patients who have been subject to polyaddictions, that is to say taking several psychotropic products at the same time.

Dependence on, also known as addiction to, one (or several) psychotropic products or the like, such as drugs or alcohol, is a chronic disorder characterized by a compulsive need for the psychotropic product or the like, loss of control regarding the quantity of product taken and the emergence of negative emotional states when the psychotropic product is not available or access to said product is prevented.

In other words therefore, an addiction is a repeated and irrepressible desire to take a psychotropic product in spite of the subject's efforts and motivation to avoid them.

In their article entitled: Development of pharmacotherapies for drug addiction: a Rosetta stone approach; Nat. Rev. Drug Discov. 2009; 8:500-515, G. F. Koob et al. reported that relapses are observed in more than 90% of patients who have been treated for dependence on a psychotropic product such as alcohol, nicotine, cocaine, heroin, opioids or the like, in the year following the end of the treatment that resulted in the weaning of said patients.

Successful prevention of relapses is essential if the aim is for the patient to attain total remission, in other words total dehabituation for a prolonged period.

However, there is at present no effective treatment targeting the symptoms at the heart of the addiction, that is to say the craving and withdrawal symptoms for the psychotropic product after a prolonged abstinence period.

The problem to be solved is therefore to propose a medicinal or pharmaceutical composition enabling a reduction in the withdrawal symptoms in patients who have been weaned, in order to avoid them relapsing or again starting to administer or ingest one or more psychotropic products.

### SUMMARY

The proposed solution therefore relates to a gaseous composition containing xenon gas for use in preventing the relapse of a patient who has been weaned from a psychotropic product which resulted in said patient becoming addicted, the xenon being administered to the patient by inhalation.

### BRIEF DESCRIPTION OF THE DRAWINGS

The invention will now be more fully explained by means of the following comparative examples given with reference to the appended figures, among which:

FIG. 1 is a diagram of the protocol concerning the effect of alcohol deprivation (EAD) in the rat;

FIG. 2 shows the effects of xenon on the consumption of alcohol after deprivation (EAD model);

FIG. 3 shows the effects of nitrous oxide (N₂O) on the consumption of alcohol after deprivation (EAD model); and

FIG. 4 is a diagram of the protocol concerning stimuli-induced alcoholic relapse in the rat;

FIG. 5 shows the effects of xenon and of nitrous oxide (N₂O) on stimuli-induced alcoholic relapse.

### DESCRIPTION OF PREFERRED EMBODIMENTS

Depending on the individual case, the inhalable gaseous composition (i.e. the gaseous medication) according to the invention can include one or more of the following characteristics:
it contains an effective volume proportion of xenon.
it contains between 5 and 70% by volume of xenon.
it contains at least 10% by volume of xenon.
it contains less than 50% by volume of xenon.
it contains less than 40% by volume of xenon, preferably less than 30% by volume of xenon.
it further contains oxygen, preferably at least 21% by volume of oxygen.
it further contains an additional compound selected from the group constituted by N₂O, Ar, Kr, He, Ne, NO, CO, H₂S and N₂.
the psychotropic product is a drug or alcohol, in particular heroin, nicotine, cannabis, cocaine or amphetamines, including derivatives of these products.
the xenon is administered to the patient by inhalation one or more times per day, during a total treatment period of several days to several years.
the patient is a human being, that is to say a man or a woman, including children, adolescents or any other group of individuals.
the gaseous xenon is mixed with a gas containing oxygen, in particular the xenon is mixed with air or an N₂/O₂ mixture.
the patient has been weaned from several psychotropic products which resulted to an addiction of said patient.

The gaseous xenon according to the invention is therefore used to manufacture an inhalable medicinal composition intended to prevent a relapse in a patient who has been weaned from a psychotropic product or several psychotropic products (i.e. in case of multiple drug addiction) which resulted in said patient becoming addicted, where said medicinal composition can include some or all of the aforesaid characteristics.

In other words, according to the invention, xenon gas is administered by inhalation to a patient, that is to say a man, a woman or a child, in order to prevent a relapse in said patient who has been weaned from a psychotropic product or several psychotropic products which resulted in said patient becoming addicted.

As a matter of fact, a dominant hypothesis in the field of addiction to drugs or other similar products or alcoholism is that the glutamatergic system is critically involved in the addictive behavior of patients, in particular during relapses as explained by J. T. Gass et al., in Glutamatergic substrates of drug addiction and alcoholism; Biochem Pharmacol; 2008; 75:218-265.

In fact, stimulation of the mGluR2/3 receptors makes it possible to block resumption of cocaine, nicotine or alcohol use, probably by reducing the release of glutamate from the presynaptic terminals, providing further evidence that it is indeed is the release of glutamate that governs relapse.

Similarly, antagonists of the group I mGluR receptors prevent the patient resuming the quest for cocaine, nicotine and alcohol, as explained by L. A. Knackstedt et al.; Glutamate and reinstatement. Curr Opin Pharmacol 2009; 9:59-64.

In studies using acamprosate, a medicament known to reduce a hyper-glutamatergic system, a reduction was noted in detrimental effects (increase in alcohol consumption) following alcohol deprivation in alcohol-dependent rats, as reported by Spanagel et al.; Acamprosate and alcohol: I. Effects on alcohol intake following alcohol deprivation in the rat. Eur J Pharmacol 1996;305:39-44.

Given that different structures in the brain contribute to the resumption of use of different classes of drugs and alcohol, the glutamatergic neuroadaptations induced by drugs and the pharmacological effects of glutamate on this resumption, which was first identified in the nucleus accumbens, can be generalized to other key brain structures, such as the ventral tegmental areas or the amygdala.

Nonetheless, the role of glutamatergic transmission in the nucleus accumbens is undeniable and important with regard to the resumption of the need to seek the drug, whatever the drug class in question may be. In other words, maintaining glutamatergic homeostasis therefore seems to be a prime pharmacotherapeutic target in the treatment of relapse in addicted patients.

Since xenon has exhibited inhibitory properties on the excitatory glutamatergic signaling pathways, firstly through its action on the N-methyl D-aspartate (NMDA) receptors, but also on the α-amino-3-hydroxy-5-methylisoazol-4-propionate (AMPA) receptors and the kainate receptors, the present invention now proposes the use of inhaled xenon to prevent or reduce the susceptibility of patients to relapse into the use of drugs and alcohol, that is to say psychotropic products causing dependence.

The document U.S.2005/0124963 teaches that a high concentration of N₂O or any other similar gaseous anesthetic agent could be used to treat alcoholism and addiction. However, xenon is not mentioned in that document. Moreover, although there are similarities between N₂O and xenon, there are also many disparities between these two gases, both as regards their anesthetic power and their analgesic power and their neuroprotective effect. Thus, the study by Valeggi et al., entitled “Xenon up regulates several genes that are not up-regulated by nitrous oxide”, J. Neurosurg. Anesthesiol. 2008, 20: 226-32, clearly demonstrates the differences in mechanism of action between these two gases. This data accounts for the differences in pharmacological effect between N₂O and xenon. Hence, an observation made for N₂O is clearly in no way extrapolable to xenon. In any case, as shown in the comparative examples below, the efficacy results obtained with xenon are very much more pronounced than those obtained with N₂O.

In general, during the treatment in the context of the present invention, the administration of xenon can be performed via a conventional means, such as a ventilator, a nebulizer or spontaneously with pre-packaged gas bottles, said means of administration being connected to a facial or nasal mask, or nasal prongs.

The period of administration is selected case by case depending on the magnitude of the withdrawal symptoms affecting the patient in question, for example, the xenon could be administered for an administration period of several minutes to several tens of minutes, even hours, for example less than one hour, up to a frequency that can reach one or more times per day or per week and over a total treatment period of one or more days, weeks, months or years, for example once a day for 6 months.

The xenon or gaseous mixture based on xenon is preferably packaged in a pressurized gas bottle or in liquid form, for example in a one or more liter (water capacity) bottle, particularly between 1 and 50 liters, and/or at a pressure lying between 2 and 300 bar.

The xenon or gaseous mixture based on xenon can be in a “ready to use” form, for example pre-mixed with oxygen, or else it can be mixed on site at the time of use, particularly with oxygen and possibly another gaseous compound, for example nitrogen.

### EXAMPLES

### Comparative Examples

The pre-clinical trials described below were carried out to show the positive effects of xenon inhalation by rats which had undergone weaning from alcohol and which are susceptible to relapse. For comparison, the effect of nitrous oxide (50%-50% vol. N₂O/O₂ mixture) will also be assessed.

Two validated models for testing treatments for alcoholic relapse in the rat were used (Sanchis-Segura and Spanagel, Behavioral assessment of drug reinforcement and addictive features in rodents: an overview, Addiction Biology 11, 2-38, 2006):
(1) Alcohol deprivation model (EAD)
(2) Stimuli-induced alcoholic relapse model

1. Alcohol Deprivation Effect Model (EAD)

The alcohol deprivation model (EAD) comprises habituating the animals to consumption of alcohol for a period of 8 weeks, followed by a period of deprivation of 2 weeks, during which alcohol was withdrawn. This cycle is repeated 8 times. After a period of deprivation, access to alcohol causes a significant but temporary increase in alcohol consumption compared to the reference level. This model is ideal for studying behavior during relapse. It was validated with nacamprosate and naltrexone. This pharmacological validation shows the predictive value of this model and allows us to characterize more precisely the presumed anti-relapse medications as well as the neurobiological mechanisms of addictive behavior.

The following experimental protocol was followed.

16 male Wistar rats have free and continuous access to 2 feeding bottles, one containing tap water and the other containing 5%, 10% and 20% ethanol (vol./ vol.) in their cages.

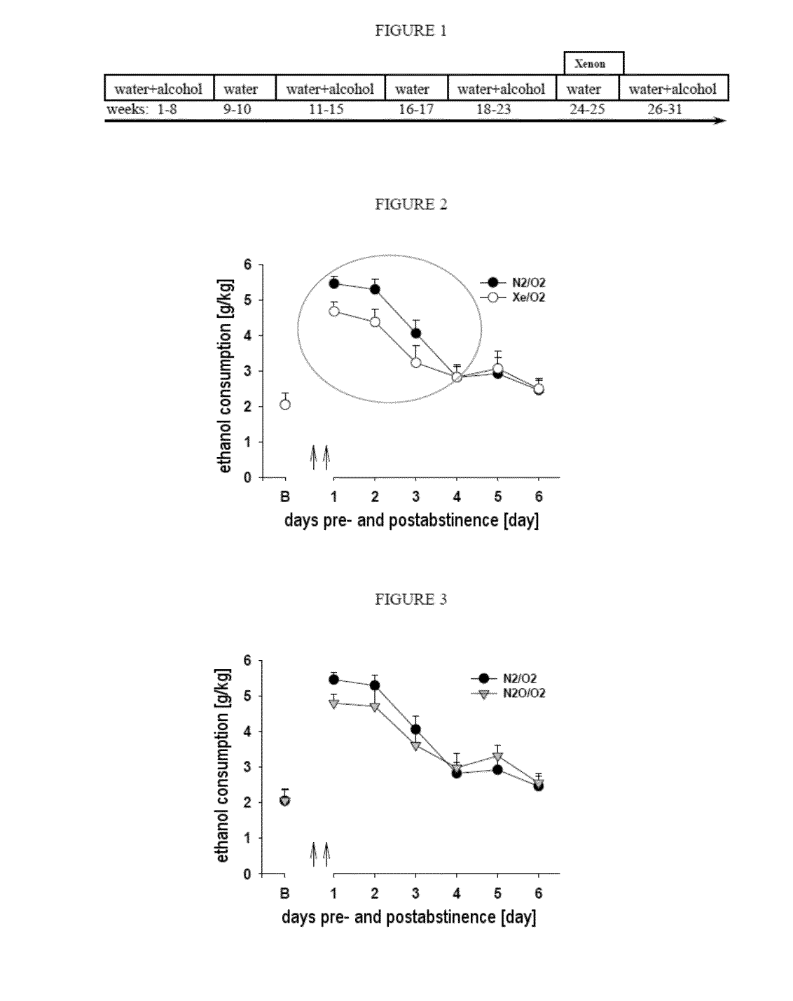

The first two-week deprivation period is introduced after 8 weeks of continuously available alcohol, as illustrated in FIG. 1.

After this deprivation period, the rats will have access to alcohol and again to at least 2 more randomly introduced periods of deprivation.

All the animals are divided into 2 groups (8 rats/group) so that for the mean baseline (week 23), the total alcohol consumption will be essentially the same in each group.

On the last day of week 25 at about 9 am, all the animals are exposed to the Xe/O₂ (50%/50%) gaseous mixture for 1 hour.

The control group is exposed to N₂/O₂ (50%/50%). The following day at 9 am all the animals are exposed for a second time to these same gaseous mixtures for 1 hour.

The exposures to the gaseous mixture are represented by two arrows in FIG. 2.

Following this last exposure, the bottles of alcohol will again be offered (post-abstinence days).

Following exposures to the different gases, the total consumption of ethanol (g/kg of body weight) is measured daily at about 10 am, for 6 days.

As can be seen in FIG. 2, following the period of abstinence, the rats greatly increased their alcohol consumption, as is typically reported in the literature with this model. Thus they pass from alcohol consumption of about 2 g/kg per day (point B in FIG. 2) to a consumption of about 5.5 g/kg one day after the period of abstinence.

Interestingly, the effect of the xenon on alcohol deprivation is reflected in a reduction in alcohol consumption following treatment with the mixture containing xenon. In fact, the consumption curve in group Xe represented by the white circles is below the control group, represented by a black circle

Calculation of the area under curve (which enables comparison of a set of points) shows a value of 14.28±1.3 g/kg*day for the Xe group less than the control group N₂/O2 which has a value of 16.34±0.9 g/kg*day. A considerable difference of two units in favor of the effect of xenon is thus observed.

For comparison, FIG. 3 shows that, under the same conditions, N₂O reduces this alcohol consumption to a much lesser extent (grey triangle).

In fact, the N₂O group results in an area under curve of 15.34±1.3 g/kg*day compared to the control group which has an area under curve of 16.34±0.9 g/kg*day. Here the difference is only 1 point, suggesting a weaker effect with the N₂O.

To summarize, the treatment by xenon inhalation will make it possible to reduce this alcohol consumption rebound thus demonstrating the anti-addictive relapse properties of inhaled xenon.

2. Model for Stimuli-Induced Alcoholic Relapse

Another animal model well-established in the literature was used to evaluate the anti-relapse effects of xenon, namely the stimuli-induced alcoholic relapse model.

This model is based on the fact that taking drugs or alcohol capable of leading to dependence is associated with a number of environmental stimuli (auditory, olfactory, visual) which are themselves likely to lead to a relapse.

This model is particularly interesting as it approximates actual conditions in humans since people who have been weaned often relapse because of environmental stimuli. The results obtained with this model are therefore particularly important.

Briefly, the experiments were carried out in conditioning chambers equipped with 2 levers allowing the distribution of water or of alcohol.

24 male Wistar rats were used. The animals are trained to self-administer ethanol at 10% by volume during the course of daily sessions lasting 30 minutes.

During the conditioning phase, the animals are trained to distinguish between the availability of alcohol and of water. Stimulus 1 (orange extract) is the signal for availability of alcohol and each pressing of lever No.1 results in conditioned stimulus No.1 (flashing light) and the immediate distribution of alcohol (30 μl). Furthermore, lever No.2 remains unused during the “alcohol sessions” throughout the experiment.

Stimulus n° 2 (anise extract) will give the signal for availability of water during the “water sessions” and each pressing of lever No.2 results in the presentation of conditioned stimulus No.2 (constant light) and the distribution of water (30 μl). The conditioning to water is used to measure the selectivity of the medicinal treatment. Lever No.1 remains unused during the “water sessions” throughout the experiment.

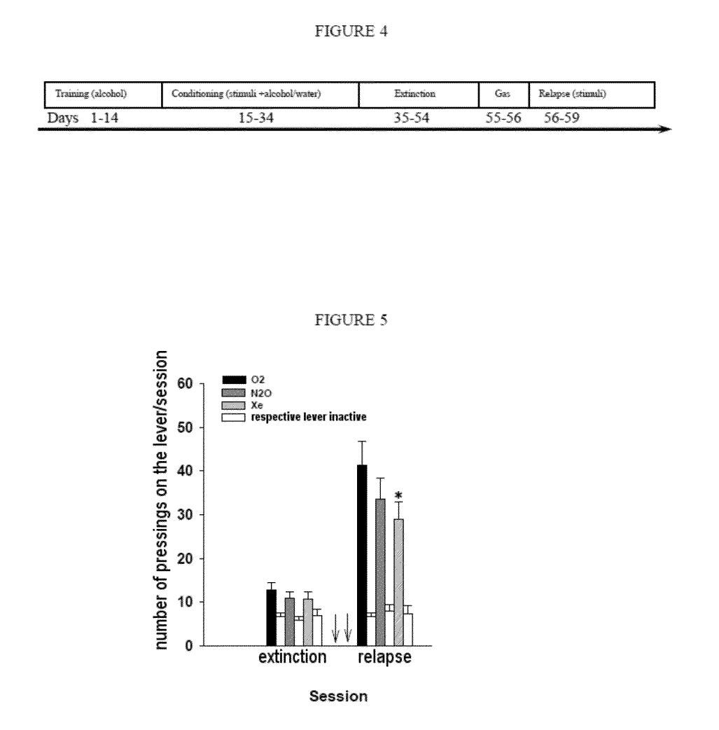

The alcohol and water sessions (one session of 30 min/day) are carried out randomly so as to obtain a total of 10 alcohol sessions and 10 water sessions (see diagram FIG. 4).

Next, the extinguishing phase begins with the presentation of the levers with no association to the olfactory stimuli. The responses to pressing the levers do not result in the provision of alcohol or water.

Finally, during the relapse phase, the rats are exposed to the same conditions as during the conditioning phase, except that the liquids (water or alcohol) are not distributed to them.

In order to test the effect of the gases, and in particular of the xenon, on the stimuli-induced alcoholic relapse, the animals are divided into three groups (8 rats/group) on the basis of their performance during the last four conditioning sessions. A 1 hour exposure to gas is carried out 24 hours (day 55) and 3 hours before the relapse test (day 56). The exposures to the gases are indicated by two arrows in FIG. 5.

As is shown in FIG. 5, the animals treated with xenon show a significant reduction in the number of presses on the lever delivering the alcohol following exposure to stimulus-1 compared to the control group breathing the N₂/O₂ 50%/50% mixture (p<0.05). Conversely, no significant effect was observed with the N₂O.

This data shows a very clear anti-relapse effect linked to the treatment with inhaled xenon but, conversely, shows that this effect is not proven with N₂O, in particular when external stimuli are associated with the taking of alcohol.

The totality of these results demonstrates the preventative anti-relapse properties of xenon in two models typically used in this condition.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1-11. (canceled)

12. A method of treating a patient using a gaseous composition containing gaseous xenon to prevent a relapse of a patient who has been weaned from at least one psychotropic product which resulted in the human being becoming addicted, said method comprising administering the gaseous composition to the patient by inhalation wherein the gaseous composition contains an effective volume proportion of xenon, lying between at least 10% and less than 50% by volume, and sufficient to prevent said relapse of said patient.

13. The method of claim 12, wherein the gaseous composition contains less than 40% by volume of xenon.

14. The method of claim 12, wherein the gaseous composition contains less than 30% by volume of xenon.

15. The method of claim 12, wherein the gaseous composition further contains oxygen.

16. The method of claim 12, wherein the gaseous composition contains 21% by volume of oxygen.

17. The method of claim 12, wherein the gaseous composition further contains an additional compound selected from the group constituted by N₂O, Ar, Kr, Ne, He, Ne, NO, CO, H₂S and N₂.

18. The method of claim 12, wherein the psychotropic product is a drug or alcohol.

19. The method of claim 12, wherein the psychotropic product is cocaine, nicotine, cannabis or amphetamines.

20. The method of claim 12, wherein the gaseous composition is administered to the patient by inhalation once or twice per day, during a total treatment period of several days to several years.

21. The method of claim 12, wherein the gaseous composition is administered to the patient one or more times per day during an inhalation period of a few minutes to one or more hours.

22. The method of claim 12, wherein the patient has been weaned from several psychotropic products which resulted to an addiction of said patient.

23. The method of claim 12, wherein the gaseous composition is provided in a gas bottle at a pressure lying between 2 and 300 bar.
